(19) United States
(12) Patent Application Publication
(10) Pub. No.: US 2005/0071005 A1
(43) Pub. Date: Mar. 31, 2005

(54) Intersomatic lumbar implant

(76) Inventors: Olivier Carli, Case Postale (FR); David N. Ryan, De Bearn (FR)

Correspondence Address: CLARK & BRODY, Suite 600, 1750 K Street, NW, Washington, DC, 20006, US

(21) Appl. No.: 10/673,163

(22) Filed: Sep. 30, 2003

(51) Int. Cl. A61F 2/44

(52) U.S. Cl. 623/17.11

(57) ABSTRACT

The purpose of the invention concerns an intersomatic implant for insertion in the intervertebral space established between two neighboring lumbar vertebrae in view of the anatomic restoration of the intervertebral space, the implant comes in the form of a cavity consisting of two sagittal walls (2, 3) connected by an anterior cross wall (5) and a posterior cross wall (4), the walls delimit an open volume for bone fill and present edges extending on one side to define a first cross side and on the other side to define a second cross side.

According to the invention, edges (10₅) of anterior wall (5) of the two cross sides present inclined profiles converging in the direction of the outer side of the anterior wall so that this anterior wall forms a projection to aid in the penetration of the intervertebral space.

The present invention involves an intersomatic implant intended to be inserted in the intervertebral space after disk excision, established between two adjacent vertebrae in order to restore a suitable intervertebral height and provide bone fusion between the aforementioned adjacent vertebrae.

More specifically, the purpose of the invention involves an intersomatic lumbar implant for insertion in an intervertebral space after disk incision, established between two adjacent lumbar vertebrae.

In the state of the technique, the insertion of an intersomatic implant in the intervertebral space between two adjacent lumbar vertebrae is known. A great many forms for such implants have been proposed in the prior art. For example, in patent FR 2 724 312, an intersomatic lumbar implant in the form of a cavity comprising two sagittal walls connected by an anterior cross wall off-center with respect to a posterior cross wall. The walls mark off an open volume intended to receive ethmoid bone favoring bone fusion between the two vertebrae. Such an implant has a relatively large support surface considering the presence of the anterior side off-center with respect to the posterior side. Moreover, it should be noted that the upper and lower edges of the walls of the cavity are equipped with notches to provide for its hold after anchoring in the bone of each adjacent vertebra. Such an implant, after introduction according to a sagittal direction is thereby designed to avoid both lateral and longitudinal migration.

Although such an implant provides a large support surface, in certain conditions, it seems difficult to introduce the implant in the intervertebral space, by a relatively narrow means of approach, in a position so that such an implant is able to provide optimum stability between the vertebrae, as this stability is absolutely necessary to obtain good bone fusion.

Therefore, the purpose of the invention is to correct the disadvantages of the state of the technique by proposing an intersomatic lumbar implant intended to be inserted between two adjacent vertebrae by a limited means of approach while allowing for easy and exact positioning in the disk space between two adjacent vertebrae.

To reach this goal, the purpose of the invention involves an intersomatic implant for insertion in the intervertebral space established between two neighboring lumbar vertebrae in view of the anatomic re-establishment of the intervertebral space, the implant comes in the form of a cavity consisting of two sagittal walls connected by an anterior cross wall and a posterior cross wall, the walls define an open volume for bone fill and present edges extending on one side to define a first cross side and on the other side, to define a second cross side. According to the invention, the edges of the anterior wall of both cross sides present inclined profiles converging in the direction of the outer side of the anterior wall so that this anterior wall forms a projection to aid in the penetration of the intervertebral space.

According to a preferred characteristic of the invention, the anterior wall is laterally off-center with respect to the posterior wall.

According to another preferred characteristic of the invention, the width of the anterior wall is greater than that of the posterior wall.

Favorably, the edges of the posterior wall of both cross sides present inclined profiles converging in the direction of the outer side of the posterior wall so as to form support edges for the circular process of the vertebrae.

Preferably, the anterior wall is connected to the sagittal walls by connecting fillets so that the aforementioned wall has a perceptibly circular cross section.

Preferably again, the posterior wall and the anterior wall each present a tapered profile in the sagittal plane.

According to a preferred characteristic of the invention, the sagittal walls present a bi-convex profile.

Preferably, the edges of the sagittal walls have notches.

According to a variant of the invention, the notches have a profile tapered in the direction of the outer sides of the sagittal walls.

Miscellaneous other characteristics result from the following description referring to the appended drawings that show, by way of non limiting examples, forms of achievement of the subject of the invention.

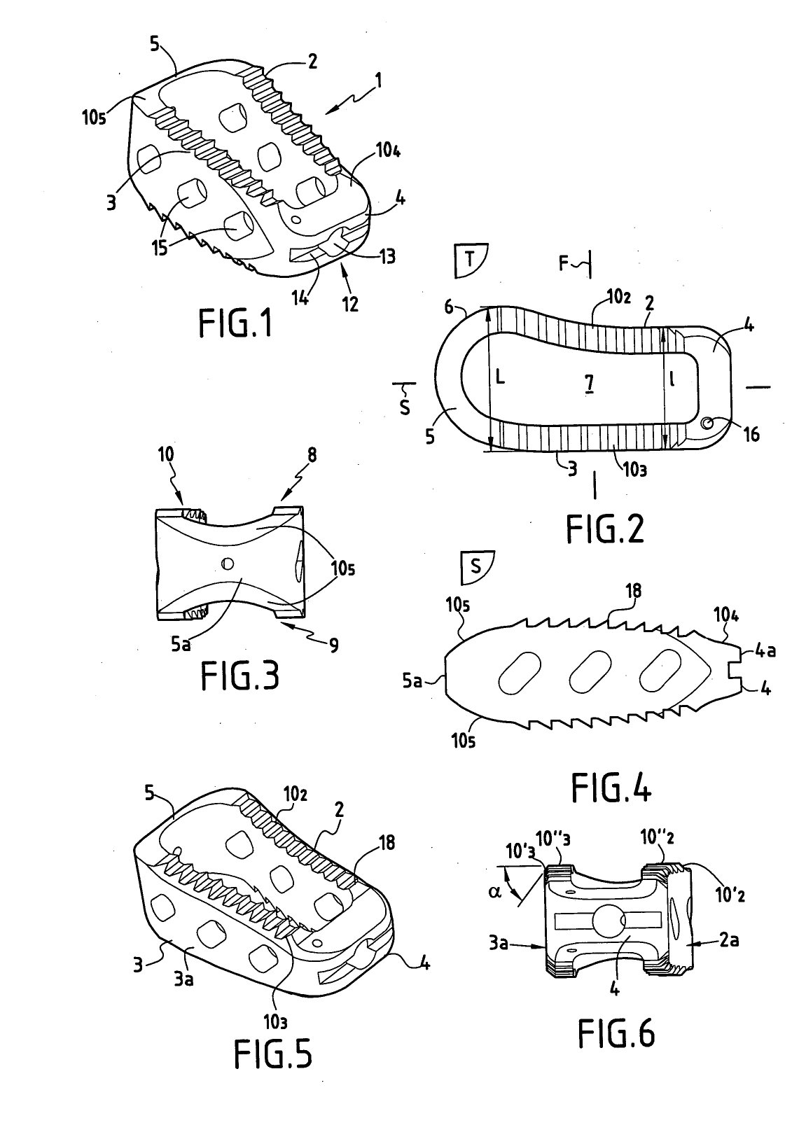

FIG. 1 is a three-quarters rear perspective of an example of an implant complying with the invention.

FIG. 2 is a top view of an implant complying with the invention.

FIG. 3 is a front view of an implant complying with the invention.

FIG. 4 is a front view in the sagittal plane of an implant complying with the invention.

FIG. 5 is a perspective view of another example of an implant complying with the invention.

FIG. 6 is a front view of the implant illustrated in FIG. 3.

As it more exactly appears in FIG. 1 to 4, the intersomatic implant complying with the invention comes in the form of a cavity 1 presenting a perceptibly parallelepiped general shape intended to be inserted in the disk space between two adjacent lumbar vertebrae. Cavity 1 consists of a first so-called inner sagittal wall 2 and a second so-called outer sagittal wall 3 perceptibly extending parallel to each other and the so-called sagittal or anteroposterior plane S. Sagittal walls 2 and 3 are connected by a so-called posterior cross wall 4 and a so-called anterior cross wall 5. Posterior cross wall 4 perceptibly extends parallel to a front plane F perpendicular to sagittal plane S while anterior wall 5 preferably has a perceptibly circular cross section in a cross plane T perpendicular to sagittal plane S. As more specifically evident in FIG. 2, anterior wall 5 is connected to sagittal walls 2, 3 by connection fillets 6 to confer anterior wall 5 with a perceptibly circular cross section in the cross section T.

According to a preferred characteristic of the invention, anterior wall 5 is off-center or laterally displaced with respect to the sagittal plane S compared with posterior wall 4. Anterior wall 5 is off-center on the side of inner sagittal wall 2.

According to a preferred characteristic of the invention, anterior wall 5 presents a width L taken in the cross plane T, which is greater than the width 1 of posterior wall 4. Anterior side 5 thereby has an enlarged shape to facilitate its insertion and the discharge of disk fragments from the anterior part to the posterior part of the implant.

Cavity 1 has an inner volume 7 defined by the inner vertical sides of walls 2 to 5 and intended to be filled with a bone complement intended for intersomatic fusion. Volume 7 opens up according to a first so-called upper cross side 8 in the illustrated example and a second so-called lower cross side 9. Walls 2 to 5 present edges 10 defining upper cross side 8 and lower cross side 9.

Advantageously, posterior cross wall 4 has a gripping means 12 such as a clearance hole 13 opening up in a groove 14 to allow for the grip and positioning of the cavity by a gripping tool. Preferably still, at least sagittal walls 2 and 3 are equipped with holes 15 communicating with volume 7 favoring the vascularisation of the bone complement.

According to the invention, anterior wall 5 has edges 105 presenting inclining profiles converging in the direction of the outer side 5a of anterior wall 5 so that this anterior wall 5 forms a projection to aid in the penetration of the intervertebral space. As more specifically indicated in FIG. 3 and 4, the edges 10₅ of anterior wall 5 opening according to two sides 8, 9 are chamfered or bevelled so that in sagittal plane S, anterior wall 5 presents a tapered profile. Inclined edges 105 allow the anterior side to remain whose height varies according to the height of the cavity. Such a tapered shape favors the insertion or penetration of the cavity inside the intervertebral space.

According to another advantageous characteristic of the invention, edges 104 of posterior wall 4 opening up according to the two cross sides 8, 9 present inclined profiles converging in the direction of outer side 4a of the posterior wall so as to form support edges for the circular process of the vertebrae. Edges 10₄ of the posterior wall are also chamfered or bevelled so that posterior wall 4 presents a tapered profile in sagittal plane S. Edges 104 thereby each present a plane support surface for the vertebrae.

According to a preferred characteristic of the invention, sagittal walls 2, 3 present a bi-convex profile in the sagittal plane. Thereby, sagittal planes 2, 3 present a convex profile in sagittal plane S, both at upper side 8 and lower side 9.

According to a preferred characteristic of the invention, posterior wall 4 is equipped with a bore 16 opening on edges 10₄ to receive a radiographic marker.

According to the invention, cavity 1 helps successfully restore the intervertebral space. The choice of the height of the anterior and posterior cross walls allows for a series of cavities with different values of lordosis re-establishment angles. In addition, the insertion of enantiomorphic complementarities, that is reverse symmetries, is foreseen in the two-cavity disk space according to the invention.

In the example illustrated in FIG. 1 to 4, edges 10₂, 10₃ respectively of sagittal walls 2, 3 are equipped with notches 18 in the upper 8 and lower sides 9 for the anchoring of the cavity. According to this example, notches 18 extend parallel to each other and to front plane F, each presenting an inclined profile in the 30 direction of the anterior wall.

In the example illustrated in FIGS. 5 and 6, notches 18 have a tapered profile in the direction of outer sides 2a, 3a of sagittal walls 2, 3. Thereby, each edge 10₂, 10₃ is chamfered or bevelled as of the outer side 2a, 3a of the sagittal walls.

Each edge 10₂, 10₃ thereby presents a chamfered part 10′₂, 10′₃ of angle α adjacent to a plane part 10″₂, 10″₃. This chamfered part 10′₂, 10′₃ equipped with notches 18 helps maintain the retention function of the cavity even if the latter is inserted at a slight angle, that is, is turned according to an anteroposterior axis.

The invention is not limited to the examples described and represented since different modifications may be made without extending beyond its scope.

1. Intersomatic implant intended for insertion in the intervertebral space defined between two neighboring lumbar vertebrae in view of anatomic restoration of the intervertebral space, the implant comes in the form of a cavity comprising of two sagittal walls (2, 3) connected by an anterior cross wall (5) and a posterior cross wall (4), the walls define an open volume (7) for bone fill and present edges (10) extending on one side to define a first cross side (8) and on the other side, to define a second cross side (9), characterized in that the edges (10₅) of the anterior wall (5) of both cross sides present inclined profiles converging in the direction of the outer side (5a) of the anterior wall so that this anterior wall forms a projection to aid in the penetration of the intervertebral space.

2. Intersomatic implant according to claim 1, characterized in that anterior wall (5) is laterally off-center with respect to posterior wall (4).

3. Intersomatic implant according to claim 2, characterized in that the width (L) of anterior wall (5) is greater than that of posterior wall (4).

4. Intersomatic implant according to claims 1, characterized in that edges (10₄) of posterior wall (4) of both cross sides present inclined profiles converging in the direction of the outer side (4a) of the posterior wall so as to form support edges for the circular process of the vertebrae.

5. Intersomatic implant according to claim 1, characterized in that anterior wall (5) is connected to sagittal walls (2, 3) by connection fillets (6) so that the aforementioned anterior wall has a perceptibly circular cross section.

6. Intersomatic implant according to claim 1, characterized in that posterior wall (4) and anterior wall (5) each present a tapered profile in sagittal plane (S).

7. Intersomatic implant according to claims 1, characterized in that sagittal walls (2, 3) present a bi-convex profile.

8. Intersomatic implant according to claims 1, characterized in that edges (10₂, 10₃) of sagittal walls (2, 3) are equipped with notches (18).

9. Intersomatic implant according to claim 8, characterized in that the notches (18) have a tapered profile in the direction of outer sides (2a, 3a) of sagittal walls (2, 3).

10. Intersomatic implant according to claim 2, characterized in that edges (10₄) of posterior wall (4) of both cross sides present inclined profiles converging in the direction of the outer side (4a) of the posterior wall so as to form support edges for the circular process of the vertebrae.

11. Intersomatic implant according to claim 3, characterized in that edges (10₄) of posterior wall (4) of both cross sides present inclined profiles converging in the direction of the outer side (4a) of the posterior wall so as to form support edges for the circular process of the vertebrae.

12. Intersomatic implant according to claim 2, characterized in that anterior wall (5) is connected to sagittal walls (2, 3) by connection fillets (6) so that the aforementioned anterior wall has a perceptibly circular cross section.

13. Intersomatic implant according to claim 4, characterized in that posterior wall (4) and anterior wall (5) each present a tapered profile in sagittal plane (S).

14. Intersomatic implant according to claim 2, characterized in that sagittal walls (2, 3) present a bi-convex profile.

15. Intersomatic implant according to claim 3, characterized in that sagittal walls (2, 3) present a bi-convex profile.

16. Intersomatic implant according to claim 4, characterized in that sagittal walls (2, 3) present a bi-convex profile.

17. Intersomatic implant according to claim 5, characterized in that sagittal walls (2, 3) present a bi-convex profile.

18. Intersomatic implant according to claim 6, characterized in that sagittal walls (2, 3) present a bi-convex profile.

19. Intersomatic implant according to claim 2, characterized in that edges (10₂, 10₃) of sagittal walls (2, 3) are equipped with notches (18).

20. Intersomatic implant according to claim 3, characterized in that edges (10₂, 10₃) of sagittal walls (2, 3) are equipped with notches (18).

21. Intersomatic implant according to claim 4, characterized in that edges (10₂, 10₃) of sagittal walls (2, 3) are equipped with notches (18).

22. Intersomatic implant according to claim 5, characterized in that edges (10₂, 10₃) of sagittal walls (2, 3) are equipped with notches (18).

23. Intersomatic implant according to claim 6, characterized in that edges (10₂, 10₃) of sagittal walls (2, 3) are equipped with notches (18).

24. Intersomatic implant according to claim 7, characterized in that edges (10₂, 10₃) of sagittal walls (2, 3) are equipped with notches (18).
